(19) United States
(12) Patent Application Publication
(10) Pub. No.: US 2024/0279396 A1
(43) Pub. Date: Aug. 22, 2024

(54) CYCLIC OLIGO(ARYLENE ETHER)S, PROCESSES FOR THEIR PREPARATION AND THEIR USE

(71) Applicant: Solvay SA, Brussels (BE)

(72) Inventors: Chantal Louis, Alpharetta, GA (US); Nicolas Treat, Alpharetta, GA (US); Joel Pollino, Johns Creek, GA (US); Simon Guelen, Chabaniere (FR); Kévin Jouvin, Voreppe (FR)

(73) Assignee: Solvay SA, Brussels (BE)

(21) Appl. No.: 18/568,708

(22) PCT Filed: May 18, 2022
(86) PCT No.: PCT/EP2022/063481

§ 371 Date: Dec. 8, 2023

Related U.S. Application Data
(60) Provisional application No. 63/277,677, filed on Nov. 10, 2021.
(60) Provisional application No. 63/208,574, filed on Jun. 9, 2021.

(30) Foreign Application Priority Data
Aug. 16, 2021 (EP) 21191569.9

(51) Int. Cl. C08G 65/40; C07D 321/00

(52) U.S. Cl. CPC C08G 65/4093; C07D 321/00; C08G 65/4012; C08G 2650/34; C08G 2650/60

(57) ABSTRACT

The invention relates to a process for the manufacture of a cyclic oligo(arylene ether) which can be a cyclic oligo(dichloromethylene arylene ether) or a cyclic oligo(arylene ether ketone); the process comprises the step of causing an aromatic compound to react with a hexachloroxylene compound in a pseudo-high dilution environment. The invention relates also to new cyclic oligo(arylene ether)s and their use for the manufacture of acyclic poly (arylene ether)s, such as PEKK, by ring-opening polymerization.

This application claims priority to the application filed on 9 Jun. 2021 in the UNITED STATES with No. 63/208,574, to the application filed on 16 Aug. 2021 in EUROPE with Nr 21191569.9, and to the application filed on 10 Nov. 2021 in the UNITED STATES with No. 63/277,677, the whole content of each of these applications being incorporated herein by reference for all purposes.

### TECHNICAL FIELD

The present invention pertains to a new process for the manufacture of cyclic oligo(dichloromethylene arylene ether)s and cyclic oligo(arylene ether ketone)s, to new cyclic oligo(dichloromethylene arylene ether)s and oligo(arylene ether ketone)s obtainable by this process and to the use of the aforementioned cyclic oligo(arylene ether ketone)s for the manufacture of acyclic poly(arylene ether ketone)s by ring-opening polymerization.

### BACKGROUND ART

Poly(arylene ether ketone)s, commonly named polyaryletherketones (PAEK), are a family of high performance thermoplastics with high-temperature stability, high mechanical strength, high crystallinity and high resistance to hydrolysis and organic solvents. Plastics that fall within this family include polyetherketoneketone (PEKK), polyetheretherketoneketone (PEEKK) and polyetherketoneetherketoneketone (PEKEKK). PAEK properties have made them desirable in many applications, including aerospace, coating and insulating materials and medical devices. PAEK, especially PEKK, is widely used in combination with long fibers to form continuous fiber composites. PEKEKK was commercialized by BASF and used to make surgical implants.

PAEK are usually produced in two ways: the nucleophilic route and the electrophilic route. Just as an example, PEKK can be prepared in solution either via nucleophilic aromatic substitution from bis(4-fluorobenzoyl)benzene and bis(4-hydroxybenzoyl)benzene monomers, or via Friedel-Crafts acylation from diphenyl ether and phthaloyl chloride monomers. Irrespectively of the conventional route by which they are made, their high crystallinity and high melt temperatures that provide many of their benefits also result in increasing their melt viscosity and decreasing their processability. So, in all applications, a compromise needs to be reached between the need for good mechanical properties (high molecular weight) and high flow (low viscosity), which is especially prejudicial in composite applications where molten polymer high flow is required to impregnate the fibers.

To overcome this issue, it has been proposed to prepare high molecular weight PAEK from low viscosity cyclic oligomer precursors by ring-opening polymerization.

CN 1 443 762 (to Changchun Applied Chemistry) describes the synthesis of cyclic oligo(arylene ether ketone)s of general formula

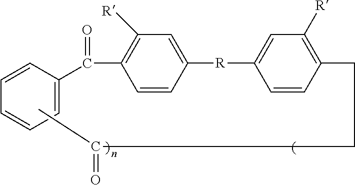

wherein
R is —O—, —CH₂—,

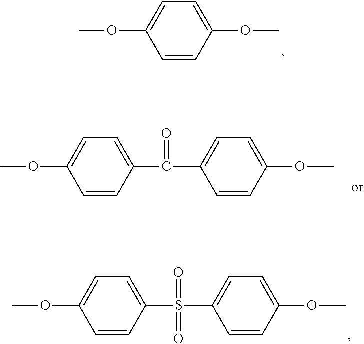

R′ is hydrogen or methyl, and n=2-10,

by causing orthophthaloyl chloride or isophthaloyl chloride monomer to react with a substituted aromatic monomer in solution in a pseudo-high-dilution environment. In examples 1 to 6, orthophthaloyl chloride was used as the acylation agent; by reacting orthophthaloyl chloride with various aromatic compounds in similar reaction conditions (with aluminum chloride as catalyst), ortho-aromatic cyclic oligomers were obtained with a yield of respectively 95%, 93%, 81%, 92%, 93% and 88%. In examples 7 to 11, isophthaloyl chloride was used instead as the acylation agent; by reacting isophthaloyl chloride with various aromatic compounds in reaction conditions similar to those previously used for orthophthaloyl chloride (notably with the same catalyst), meta-aromatic cyclic oligomers were obtained with a much lower yield, namely respectively 64%, 62%, 65%, 61% and 60%. The heavy decrease in yield when switching from orthophthaloyl chloride to isophthaloyl chloride was reasonably expected by the skilled person, because meta-phenylene rings are known to form cyclics less readily due to poor bond geometry. It is also understood by the skilled person that p-phenylene rings of cyclic oligomers would be even more difficult to access due to poor bond geometry; so, unsurprisingly, CN 1 443 762 remains totally silent about any process that would be able to produce such para-aromatic cyclic oligomers.

When replicating Changchun's process, the Applicant has also faced with two more issues. Firstly, the Applicant observed that the acylation reaction resulted in the undesirable formation of a substantial amount of insoluble solid by-product, which had to be removed by filtration. Then, while CN 1 443 762 teaches that the cyclic oligomers prepared by Changchun's process would be mainly dimers and trimers (without providing any experimental result on the size distribution of the cyclic oligomers prepared in any of its 32 examples), the Applicant, which determined the composition of so-prepared cyclic oligomers noted that these ones, although containing predominantly cyclic trimers, were completely free of cyclic dimers. Yet, as strongly suggested by Changchun itself, such cyclic dimers have a somewhat lower melt viscosity than their higher-size homologues (trimers, tetramers, etc.), which enables the ring-opening polymerization to be carried out at a lower temperature, provides a wider processing window for the preparation of composite materials, and makes the ring-forming reaction route have an important application prospect in the field of preparing high-performance composite materials.

There is a need for providing a process for the manufacture of cyclic oligo(arylene ether ketone) products with a higher yield than the one obtained by Changchun, in particular when meta-aromatic cyclic oligo(arylene ether ketone)s are desired.

There is also need for providing a process capable of manufacturing para-aromatic cyclic oligo(arylene ether ketone)s, desirably with a high yield and selectivity.

There is also need for providing a process for the manufacture of cyclic oligo(arylene ether ketone)s which does not generate a substantial amount of insoluble by-products.

Finally, there is a need for providing a process for the manufacture of cyclic oligo(arylene ether ketone)s including cyclic dimers, desirably a process wherein such cyclic dimers are produced with a high yield and selectivity.

### Main Aspects of the Invention

These needs, and still other ones, are met by a process P¹ for the manufacture of a cyclic oligo(arylene ether) of formula (I)

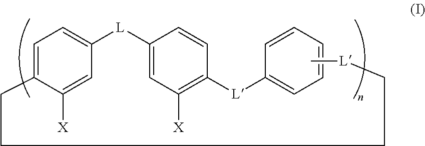

wherein
X, which may be the same or different at each occurrence, is hydrogen or methyl,
L, which may be the same or different at each occurrence, is a divalent moiety selected from the group consisting of
—O—, —CX₂— with X as previously defined,

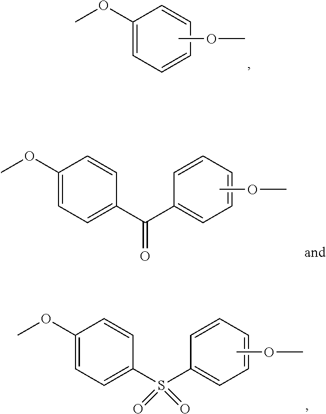

L′, which is the same at each occurrence, is a —CCl₂— or

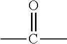

divalent moiety, and
n is an integer ranging from 2 to 20,
   said process comprising a step S¹ of causing an aromatic compound A of formula (II)

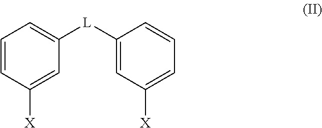

wherein X and L are as defined for the cyclic oligo(arylene ether) of formula (I), to react with a hexachloroxylene compound of formula (III)

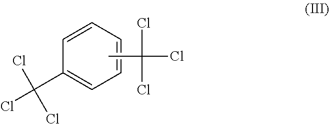

in a reaction medium comprising a solvent S capable of dissolving the cyclic oligo(arylene ether), the aromatic compound A and the hexachloroxylene compound, wherein the reaction medium is a pseudo-high-dilution environment.

Advantageously, the aromatic compound A and/or the hexachloroxylene compound are introduced progressively in the reaction medium.

The reaction between the aromatic compound A and the hexachloroxylene compound which takes place during the step S¹ is typically a Friedel-Crafts alkylation reaction.

As reaction product of the reaction which takes place during the step S¹ between the aromatic compound A and the hexachloroxylene compound, a cyclic oligo(dichloromethylene arylene ether) of formula (IV) is obtained

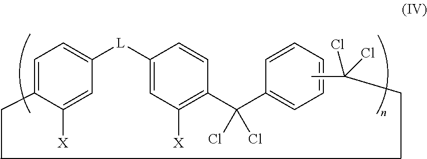

wherein X, L and n are as defined for the cyclic oligo(arylene ether) of formula (I).

The process P¹ according to the present invention may further comprise a step S², wherein the oligo(dichloromethylene arylene ether) of formula (IV) obtained during the step S¹ is hydrolyzed, so as to obtain a cyclic oligo(arylene ether ketone) of formula (V)

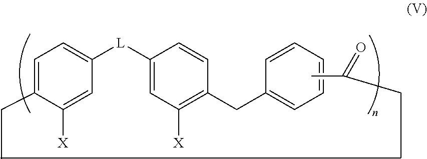

wherein X, L and n are as defined for the cyclic oligo(arylene ether) of formula (I).

The present invention concerns also a process P² for the manufacture of an acyclic poly(arylene ether) comprising m repeat units of formula (VI)

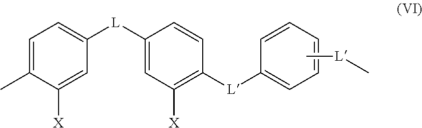

wherein m is an integer which is greater than 5 times n and wherein X, L, L′ and n are as defined for the cyclic oligo(arylene ether) of formula (I), said process comprising the steps of:
manufacturing the cyclic oligo(arylene ether) of formula (I) by the process P¹, and
causing the cyclic oligo(arylene ether) of formula (I) to undergo a ring-opening polymerization, so as to obtain the poly(arylene ether) comprising m repeat units of formula (VI).

In the process P², when the cyclic oligo(arylene ether) of formula (I) is the cyclic oligo(dichloromethylene arylene ether) of formula (IV), the acyclic poly(arylene ether) comprising m repeat units of formula (VI) is an acyclic poly(dichloromethylene arylene ether) comprising m repeat units of formula (VII)

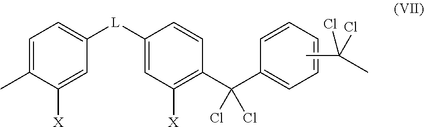

wherein m is as defined for the acyclic poly(arylene ether) of formula (VI), that is to say that m is an integer which is greater than 5 times n, and X, L and n are as defined for the cyclic oligo(arylene ether) of formula (I). On the other hand, when the cyclic oligo(arylene ether) of formula (I) is the cyclic oligo(arylene ether ketone) of formula (V), the acyclic poly(arylene ether) comprising m repeat units of formula (VI) is an acyclic poly(arylene ether ketone) comprising m repeat units of formula (VIII)

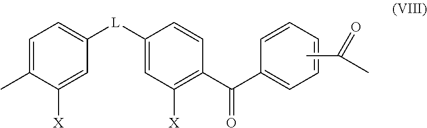

wherein m is as defined for the acyclic poly(arylene ether) of formula (VI), that is to say that m is an integer which is greater than 5 times n, and X, L and n are as defined for the cyclic oligo(arylene ether) of formula (I).

In a particular embodiment of high industrial importance of the process P², the manufactured acyclic poly(arylene ether ketone) comprising m repeat units of formula (VIII) is comprised in a composite material. In this embodiment, the process P² comprises the steps of:
manufacturing the cyclic oligo(arylene ether ketone) of formula (V) by the process P¹,
impregnating a continuous fiber with the cyclic oligo(arylene ether ketone) of formula (V), so as to form a pre-composite material, and
causing the cyclic oligo(arylene ether ketone) of formula (V) which is comprised in the pre-composite material to undergo a ring-opening polymerization, so as to form the composite material comprising the acyclic poly(arylene ether ketone) comprising m repeat units of formula (VIII).

The present invention concerns also the cyclic oligo(dichloromethylene arylene ether) of formula (IV) as such.

The present invention concerns also a method M¹ for the manufacture of the cyclic oligo(arylene ether ketone) of formula (V), said method M¹ comprising the hydrolysis of the cyclic oligo(dichloromethylene arylene ether) of formula (IV) into the cyclic oligo(arylene ether ketone) of formula (V).

The present invention concerns also a use U¹ of the cyclic oligo(dichloromethylene arylene ether) of formula (IV) for the manufacture of a composite material comprising the poly(arylene ether ketone) comprising m repeat units of formula (VIII) and a continuous fiber.

The present invention concerns also a para-aromatic cyclic oligo(arylene ether) of formula (IX)

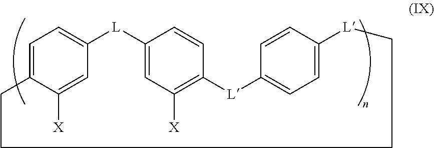

as such, wherein X, L, L′ and n are as defined for the cyclic oligo(arylene ether) of formula (I).

The para-aromatic cyclic oligo(arylene ether) of formula (IX) can be a para-aromatic cyclic oligo(dichloromethylene arylene ether) of formula (X)

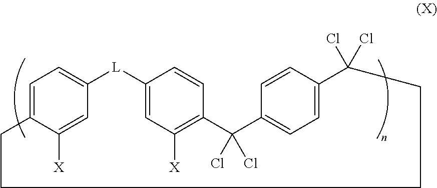

wherein X, L and n are as defined for the cyclic oligo(arylene ether) of formula (I), or a para-aromatic cyclic oligo(arylene ether ketone) of formula (XI)

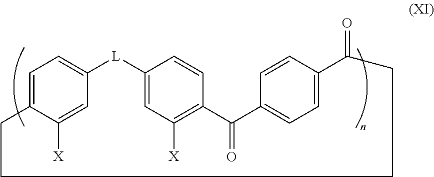

wherein X, L and n are also as defined for the cyclic oligo(arylene ether) of formula (I).

The para-aromatic cyclic oligo(arylene ether)s of formulae (IX), (X) and (XI) can be manufactured by the process P¹, using α,α,α,α′,α′,α′-hexachloro-p-xylene, also named hexachloroparaxylol or 1,4-bis(trichloromethyl)benzene, as the hexachloroxylene compound. The para-aromatic cyclic oligo(arylene ether ketone) of formula (XI) can be manufactured by the method M¹, wherein the para-aromatic cyclic oligo(dichloromethylene arylene ether) of formula (X) is hydrolyzed into the para-aromatic cyclic oligo(arylene ether ketone) of formula (XI).

The invention concerns also a method M² for the manufacture of an acyclic poly(arylene ether) comprising m repeat units of formula (XII)

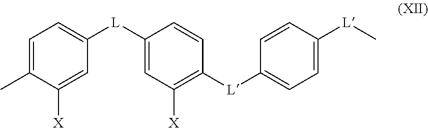

wherein m is as defined for the acyclic poly(arylene ether) of formula (VI), that is to say that m is an integer which is greater than 5 times n, and wherein X, L, L′ and n are as defined for the cyclic oligo(arylene ether) of formula (I), said method comprising the ring-opening polymerization of the para-aromatic cyclic oligo(arylene ether) of formula (IX).

In the method M², when the para-aromatic cyclic oligo(arylene ether) of formula (IX) is the para-aromatic cyclic oligo(dichloromethylene arylene ether) of formula (X), the acyclic poly(arylene ether) comprising m repeat units of formula (XII) is an acyclic poly(dichloromethylene arylene ether) comprising m repeat units of formula (XIII)

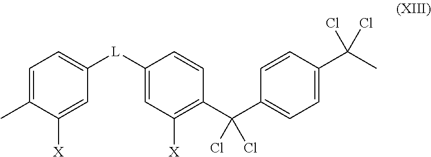

wherein m is as defined for the acyclic poly(arylene ether) of formula (VI), that is to say that m is an integer which is greater than 5 times n, and X, L and n are as defined for the cyclic oligo(arylene ether) of formula (I). On the other hand, when the para-aromatic cyclic oligo(arylene ether) of formula (IX) is the para-aromatic cyclic oligo(arylene ether ketone) of formula (XI), the acyclic poly(arylene ether) comprising m repeat units of formula (XII) is an acyclic poly(arylene ether ketone) comprising m repeat units of formula (XIV)

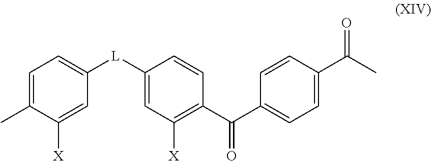

wherein m is as defined for the acyclic poly(arylene ether) of formula (VI), that is to say that m is an integer which is greater than 5 times n, and X, L and n are as defined for the cyclic oligo(arylene ether) of formula (I); the case being, the method M² can further comprise the manufacture of the para-aromatic cyclic oligo(arylene ether ketone) of formula (XI) by the method M¹, that is to say the hydrolysis of the para-aromatic cyclic oligo(dichloromethylene arylene ether) of formula (X) into the para-aromatic cyclic oligo(arylene ether ketone) of formula (XI).

The present invention concerns also a use U² of the para-aromatic cyclic oligo(arylene ether) of formula (IX) for the manufacture of a composite material comprising the acyclic poly(arylene ether ketone) comprising m repeat units of formula (XIV) and a continuous fiber.

The present invention concerns also a meta-aromatic cyclic di(arylene ether) of formula (XV)

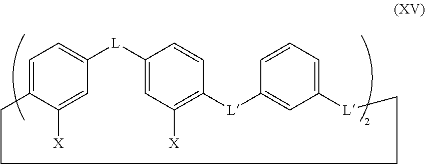

as such, wherein X, L and L′ are as defined for the cyclic oligo(arylene ether) of formula (I).

The meta-aromatic cyclic di(arylene ether) of formula (XV) can be a meta-aromatic cyclic di(dichloromethylene arylene ether) of formula (XVI)

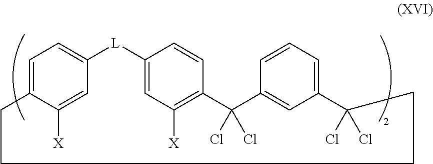

wherein X and L are as defined for the cyclic oligo(arylene ether) of formula (I), or a meta-aromatic cyclic di(arylene ether ketone) of formula (XVII)

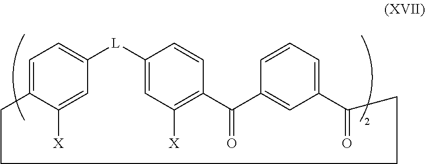

wherein X and L are also as defined for the cyclic oligo(arylene ether) of formula (I).

The meta-aromatic cyclic di(arylene ether)s of formulae (XV), (XVI) and (XVII) can be manufactured by the process P¹, generally in mixtures further comprising meta-aromatic cyclic oligo(arylene ether)s having more than 2 repeat units, for example from 3 up to 6, 10 or 20 repeat units. The meta-aromatic cyclic di(arylene ether ketone) of formula (XVII) can be manufactured by the method M¹, wherein the meta-aromatic cyclic di(dichloromethylene arylene ether) of formula (XVI) is hydrolyzed into the meta-aromatic cyclic di(arylene ether ketone) of formula (XVII).

The invention concerns also a method M³ for the manufacture of an acyclic poly(arylene ether) comprising m′ repeat units of formula (XVIII)

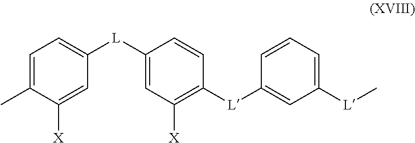

wherein m′ is an integer which is greater than 10, and wherein X, L and L′ are as defined for the cyclic oligo(arylene ether) of formula (I), said method comprising the ring-opening polymerization of the meta-aromatic cyclic di(arylene ether) of formula (XV).

In the method M³, when the meta-aromatic cyclic di(arylene ether) of formula (XV) is the meta-aromatic cyclic di(dichloromethylene arylene ether) of formula (XVI), the acyclic poly(arylene ether) comprising m′ repeat units of formula (XVIII) is an acyclic poly(dichloromethylene arylene ether) comprising m′ repeat units of formula (XIX)

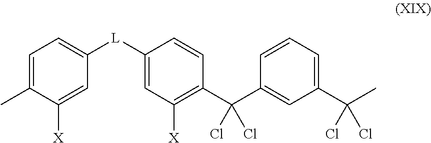

wherein m′ is as defined for the acyclic poly(arylene ether) of formula (XVIII), that is to say that m′ is an integer which is greater than 10, and X and L are as defined for the cyclic oligo(arylene ether) of formula (I). On the other hand, when the meta-aromatic cyclic oligo(arylene ether) of formula (XV) is the meta-aromatic cyclic di(arylene ether ketone) of formula (XVII), the acyclic poly(arylene ether) comprising m′ repeat units of formula (XVIII) is an acyclic poly(arylene ether ketone) comprising m′ repeat units of formula (XX)

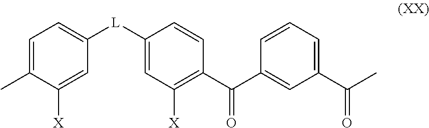

wherein m′ is as defined for the acyclic poly(arylene ether) of formula (XVIII), that is to say that m′ is an integer which is greater than 10, and X and L are as defined for the cyclic oligo(arylene ether) of formula (I); the case being, the method M³ can further comprise the manufacture of the meta-aromatic cyclic di(arylene ether ketone) of formula (XVII) by the method M¹, that is to say the hydrolysis of the meta-aromatic cyclic di(dichloromethylene arylene ether) of formula (XVI) into the meta-aromatic cyclic di(arylene ether ketone) of formula (XVII).

The present invention concerns also a use U³ of the meta-aromatic cyclic di(arylene ether) of formula (XV) for the manufacture of a composite material comprising the acyclic poly(arylene ether ketone) comprising m′ repeat units of formula (XX) and a continuous fiber.

A special embodiment of the invention concerns a method M⁴ for the manufacture of the acyclic poly(arylene ether ketone) comprising m repeat units of formula (VIII), said method M⁴ comprising the hydrolysis of the acyclic poly(dichloromethylene arylene ether) comprising m repeat units of formula (VII) into the acyclic poly(arylene ether ketone) comprising m repeat units of formula (VIII). In the method M⁴, the acyclic poly(dichloromethylene arylene ether) comprising m repeat units of formula (VII) is advantageously manufactured by causing the cyclic oligo(dichloromethylene arylene ether) of formula (IV) to undergo a ring-opening polymerization.

### DETAILED DESCRIPTION OF THE INVENTION

In the cyclic oligo(arylene ether) of formula (I), the two L′ can be in ortho positions with respect to each other.

Nevertheless, PAEK of most industrial importance are generally manufactured from a cyclic oligo(arylene ether) of formula (I) wherein the two L′ are in meta positions with respect to each other, that is to say a meta-aromatic cyclic oligo(arylene ether) of formula (XXI)

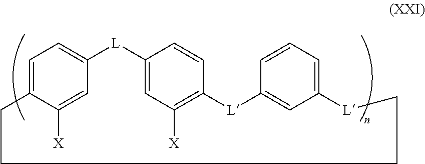

wherein X, L, L′ and n are as defined for the cyclic oligo(arylene ether) of formula (I), and/or
from a cyclic oligo(arylene ether) of formula (I) wherein the two L′ are in para positions with respect to each other, that is to say the para-aromatic cyclic oligo(arylene ether) of formula (IX) as previously described.

In the cyclic oligo(arylene ether) of formula (I) and in all the other compounds and moieties described in the present document which comprise X, X is preferably hydrogen.

In the cyclic oligo(arylene ether) of formula (I) and in all the other compounds and moieties described in the present document which comprise L, a first preferred selection for L consists in selecting L from the group consisting of
—O—, —CH₂—,

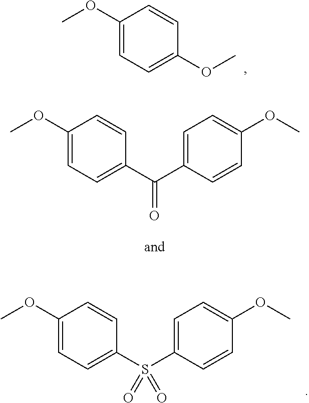

Another preferred selection for L consists in selecting L from the group consisting of
—O—,

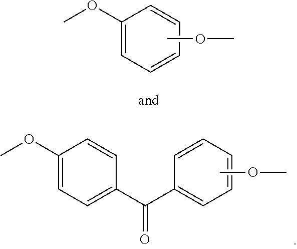

More preferably, L is a divalent moiety selected from the group consisting of
—O—,

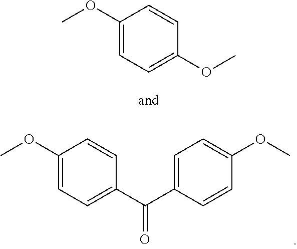

Still more preferably, L is —O—.

As already indicated, L′, which is the same at each occurrence, is a —CCl₂— (dichloromethylene) or

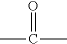

(carbonyl) divalent moiety.

n ranges preferably from 2 to 10. More preferably, n ranges from 2 to 8; for example, n can be 2, 3, 4, 5, 6, 7 or 8. Still more preferably, n ranges from 2 to 6 and even still more preferably n ranges from 2 to 5. Of particular interest are the cyclic oligo(arylene ether)s of formulae (I), (IV), (V), (IX), (X) and (XI) wherein n=2, as well as the previously described cyclic di(arylene ether)s of formulae (XV), (XVI) and (XVII).

Preferred and of high industrial importance are the cyclic oligo(dichloromethylene arylene ether)s of formulae (XXII) and (XXIII), and the cyclic oligo(arylene ether ketone)s of formula (XXIV) and (XXV)

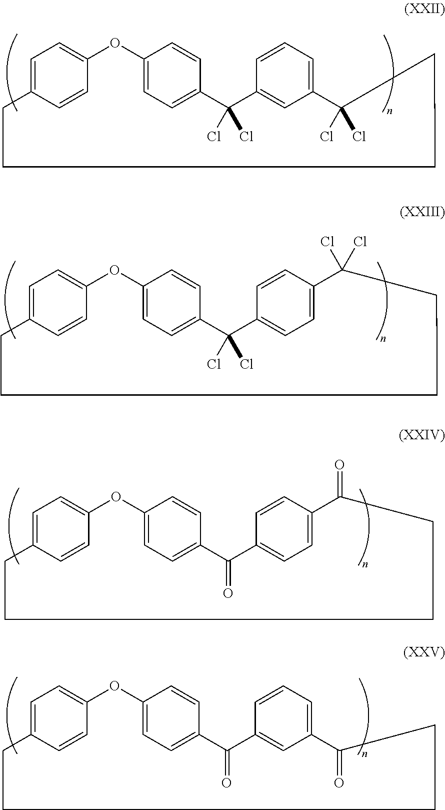

wherein n ranges from 2 to 20, preferably from 2 to 10, more preferably from 2 to 8, still more preferably from 2 to 6 and even still more preferably from 2 to 5, in particular the cyclic compounds of formulae (XXII), (XXIII), (XXIV) and (XXV) wherein n=2.

As examples of suitable aromatic compounds A, it can be notably cited phenoxybenzene (commonly referred to as diphenyl ether), 1,2-diphenoxybenzene, 1,3-diphenoxybenzene, 1,4-diphenoxybenzene, 4,4′-phenoxybenzophenone, 4,4′-(3-methylphenoxy)-benzophenone, 4,4′-phenoxydiphenylsulfone and 4,4′-(3-methylphenoxy)-diphenylsulfone.

In the aromatic compound A of formula (II), L and X meet the same features and preferences as above specified for the cyclic oligo(arylene ether) of formula (I). So, the most preferred aromatic compound A is phenoxybenzene.

The hexachloroxylene compound of formula (III) can be selected from the group consisting of 1,2-bis(trichloromethyl)benzene, 1,3-bis(trichloromethyl)benzene, 1,4-bis(trichloromethyl)benzene and mixtures thereof. It is preferably selected from the group consisting of 1,3-bis(trichloromethyl)benzene, 1,4-bis(trichloromethyl)benzene and mixtures thereof. Excellent results were obtained when using 1,3-bis(trichloromethyl)benzene (also named hexachloro-m-xylene or hexachlorometaxylol) as the hexachloroxylene compound. Excellent results were also obtained when using 1,4-bis(trichloromethyl)benzene (also named hexachloro-p-xylene or hexachloroparaxylol) as the hexachloroxylene compound.

The total amount of the aromatic compound A which is introduced in the reaction medium during the step S¹ does not generally exceed 1 mole by liter of the solvent, wherein the volume of the solvent is measured at 25° C. and 1 atm (101325 Pa). It is preferably of at most 0.2 mol/l, more preferably of at most 50 mmol/l and still more preferably of at most 30 mmol/l. Besides, it is generally of at least 0.1 mmol/l, preferably of at least 1 mmol/l, more preferably of at least 5 mmol/l and still more preferably of at least 10 mmol/l.

The total amount of the hexachloroxylene compound which is introduced in the reaction medium during the step S¹ does not generally exceed 1 mole by liter of the solvent, wherein the volume of the solvent is measured at 25° C. and 1 atm (101325 Pa). It is preferably of at most 0.2 mol/l, more preferably of at most 50 mmol/l and still more preferably of at most 30 mmol/l. Besides, it is generally of at least 0.1 mmol/l, preferably of at least 1 mmol/l, more preferably of at least 5 mmol/l and still more preferably of at least 10 mmol/l.

The total combined amount of the aromatic compound A and of the hexachloroxylene compound which are introduced in the reaction medium during the step S¹ does not generally exceed 2 mole by liter of the solvent, wherein the volume of the solvent is measured at 25° C. and 1 atm (101325 Pa). It is preferably of at most 0.4 mol/l, more preferably of at most 100 mmol/l and still more preferably of at most 60 mmol/l. Besides, it is generally of at least 0.2 mmol/l, preferably of at least 2 mmol/l, more preferably of at least 10 mmol/l and still more preferably of at least 20 mmol/l.

The ratio of the total number of moles of the aromatic compound A which is introduced in the reaction medium during the step S¹ to the ratio of the total number of moles of the hexachloroxylene compound which is introduced in the reaction medium during the step S¹ ranges generally from 0.1 to 10, preferably from 0.5 to 2, more preferably from 0.90 to 1.10, still more preferably from 0.98 to 1.02. The most preferably, during the S¹, the aromatic compound A and the hexachloroxylene compound are introduced in equimolar amounts in the reaction medium.

The reaction medium is advantageously commonly referred to as a “pseudo-high-dilution environment”. A pseudo-high-dilution environment makes it possible to obtain the desired reaction product of the step S¹, viz. the cyclic oligo(dichloromethylene arylene ether) of formula (IV), with a high selectivity. A pseudo-high-dilution environment can be created by introducing during the step S¹ the aromatic compound A and/or the hexachloroxylene compound progressively in the reaction medium, as advantageously achieved in accordance with the present invention.

In accordance with the present invention, the aromatic compound A and/or the hexachloroxylene compound are advantageously introduced progressively in the reaction medium.

The aromatic compound A and/or the hexachloroxylene compound can be introduced through repeated injections, dropwise or continuously in the reaction medium. Preferably, the aromatic compound A and/or the hexachloroxylene compound are introduced dropwise or continuously in the reaction medium. More preferably, the aromatic compound A and/or the hexachloroxylene compound are introduced continuously in the reaction medium. Still more preferably, the aromatic compound A and/or the hexachloroxylene compound are introduced continuously in the reaction medium at a constant introduction rate.

Let D¹ be the duration of the period of time I¹ enveloping the introduction of the whole amount of the aromatic compound A and the whole amount of the hexachloroxylene compound in the reaction medium. I¹ starts with the beginning of the introduction of at least part of the amount of the aromatic compound A and/or at least part of the amount of the hexachloroxylene compound in the reaction medium. I¹ finishes with the completion of the introduction the whole amount of the aromatic compound A and the whole amount of the hexachloroxylene compound in the reaction medium.

Let us divide the period of time I¹ in k parts of equal duration D¹/k, wherein k is 8, 16, 32, 64, 128, 256 or 512.

Let r_{avg.A}, r²_{avg.A}, . . . , r^{k}_{avg.A} represent the k average introduction rates of the aromatic compound A respectively during the 1^{st}, 2^{nd}, . . . and k^{th} part of I¹, and let r_{avg.B}, r²_{avg.B}, . . . , r^{k}_{avg.B} represent the k average introduction rates of the hexachloroxylene compound respectively during the 1^{st}, 2^{nd}, . . . and k^{th} part of I¹.

Advantageously, at least half of, preferably at least three-quarters of, more preferably at least seven-eighths of and still more preferably all the k average introduction rates of the aromatic compound A r_{avg.A}, r²_{avg.A}, . . . r^{k}_{avg.A} do not exceed a certain value r_{A}^{max}. Likewise, advantageously at least half of, preferably at least three-quarters of, more preferably at least seven-eighths of and still more preferably all the k average introduction rates of the hexachloroxylene compound r_{avg.B}, r²_{avg.B}, . . . r^{k}_{avg.B} do not exceed a certain value r_{B}^{max}.

r_{A}^{max} and/or r_{B}^{max} can be 10, 5, 4, 3, 2.5, 2, 1, 0.5 or 0.2 mmol/(l·h). r_{A}^{max} and/or r_{B}^{max} can also be even lower, e.g. 0.1 or 0.01 mmol/(l·h). The lower r_{A}^{max} and r_{B}^{max} are, the more the reaction medium can be seen as a pseudo-high-dilution environment, favoring a high selectivity. However, the lower r_{A}^{max} and/or r_{B}^{max}, the lower the amount of the reagents which are susceptible of forming the desired cyclic oligo(dichloromethylene arylene ether). So, to achieve a high yield, a compromise needs to be found, which compromise can be often achieved by specifying a r_{A}^{max} and/or r_{B}^{max} value in the range of from 0.5 to 5, e.g. 2 or 3.

Good results are obtained when I¹ is divided in k=32 or more parts, and either all the k average introduction rates r_{avg.A}, r²_{avg.A}, . . . r^{k}_{avg.A}, of the aromatic compound A, equal to or different from each other, or all the k average introduction rates r_{avg.B}, r²_{avg.B}, . . . r^{k}_{avg.B}, of the hexachloroxylene compound, equal to or different from each other, are from 0.5 to 5 mmol/(l·h), especially from 2 to 3 mmol/(l·h).

Very good results were obtained when I¹ was divided in k=32 or more parts, and all the k average introduction rates r_{avg.A}, r²_{avg.A}, . . . r^{k}_{avg.A}, of the aromatic compound A, equal to or different from each other, and all the k average introduction rates r_{avg.B}, r²_{avg.B}, . . . r^{k}_{avg.B}, of the hexachloroxylene compound, equal to or different from each other, were from 1 to 5 mmol/(l·h), especially from 2 to 3 mmol/(l·h).

Excellent results are obtained when, during the whole course of the period of time I¹, equimolar amounts of the aromatic compound A and the hexachloroxylene compound are introduced continuously in the reaction medium, at one single, constant rate in the range of from 1 to 5 mmol/(l·h), especially from 2 to 3 mmol/(l·h), that is to say that, during the whole course of the period of time I¹. (i) the introduction rate of the aromatic compound A (r_{A}) is kept constant at one single value to be chosen in the range of from 1 to 5 mmol/(l·h), especially from 2 to 3 mmol/(l·h) and (ii) the introduction rate of the hexachloroxylene compound B (r_{B}) is also kept constant at the same, single value as the one chosen for r_{A}.

Once I¹ finishes, the reaction between the aromatic compound A and the hexachloroxylene compound may be continued for at least 10 min, at least 30 min, at least 1 h, at least 2 h or even more to ensure the highest achievable conversion into the cyclic oligo(dichloromethylene arylene ether) of formula (IV). Alternatively, immediately afterwards or less than 10 min after I¹ finishes, it can be proceeded with a possible subsequent step, which may be for example the concentration and purification of the cyclic oligo(dichloromethylene arylene ether) of formula (IV) when said cyclic oligo(dichloromethylene arylene ether) is the desired reaction product, or the hydrolysis of the cyclic oligo(dichloromethylene arylene ether) of formula (IV) when the cyclic oligo(arylene ether ketone) of formula (V) is the desired product.

The whole amount of the solvent S is advantageously present in the reaction medium before the introduction of the aromatic compound A and the introduction of the hexachloroxylene compound have started.

The aromatic compound A and the hexachloroxylene compound reagents are advantageously substantially dissolved, preferably completely dissolved, in the reaction medium comprising the solvent S. The cyclic oligo(dichloromethylene arylene ether) of formula (IV), viz. the reaction product of the reaction which takes place during the step S¹ between the aromatic compound A and the hexachloroxylene compound, is also advantageously substantially dissolved, preferably completely dissolved, in the reaction medium comprising the solvent S. Likewise, the cyclic oligo(arylene ether ketone) of formula (V), viz. the product of the hydrolysis of the cyclic oligo(dichloromethylene arylene ether) of formula (IV), is also advantageously substantially dissolved, preferably completely dissolved, in the reaction medium comprising the solvent S.

The solvent S is generally chosen from the group consisting of carbon disulfide, nitroaromatics, nitroalkanes, chloroaromatics, chloroalkenes and chloroalkanes. As nitroaromatics, it can be cited mononitrobenzene, 2-nitrotoluene and 3-nitrotoluene. As nitroalkanes, it can be cited C₁-C₄ alkanes, such as nitromethane, nitroethane, 1-nitropropane and 2-nitropropane. As chloroaromatics, it can be cited monochlorobenzene, o-, m- and p-dichlorobenzenes, and o-, m- and p-chlorotoluenes. As chloroalkenes, it can be cited cis- and trans-1,2-dichloroethenes, and cis- and trans-1,2,3-trichloro-1-propenes.

In a first preferred embodiment, the solvent S is a chloroalkane, more preferably a chloroalkane which is free of any carbon atom to which 2 or more than 2 chlorine would be attached. The chloroalkane has generally from 1 to 10 carbon atoms, preferably from 2 to 6 carbon atoms, more preferably from 2 to 4 carbon atoms, still more preferably 2 carbon atoms. As chloroalkanes, it can be cited dichloromethane, 1,1-dichloroethane, dichloropropanes such as 1,2- and 1,3-dichloropropanes, trichlorobutanes such as 1,2,3- and 1,2-4-trichlorobutanes and the chloroalkanes of formula CH₂Cl—(CHCl)ⱼ—CH₂Cl wherein j is an integer from 0 to 8, especially 1,2-dichloroethane, 1,2,3-trichlororopane and 1,2,3,4-tetrachlorobutane. Good results were obtained when using 1,2-dichloroethane as the solvent S.

In another preferred embodiment, the solvent S is a chloroaromatic. The chloroaromatic is preferably selected from the group consisting of monochlorobenzene, o-, m- and p-dichlorobenzenes, o-, m- and p-monochlorotoluenes and dichlorotoluenes. It is more preferably selected from the group consisting of monochlorobenzene, o-dichlorobenzene, m-dichlorobenzene and p-dichlorobenzene. Good results are obtained when using o-dichlorobenzene as the solvent S.

The reaction medium can comprise a catalyst. The cyclization reaction rate between the aromatic compound A of formula (II) and the hexachloroxylene compound of formula (III) can be increased by the catalyst. The catalyst may be in anhydrous form or in a hydrated form; in accordance with the invention, the catalyst is preferably anhydrous. The catalyst can be a zeolite (in particular a zeolite beta, Y or ZSM-5), a heteropolyacid such as 12-tungstophosphoric acid (H₃PW₁₂O₄₀) or an oxide of an element M, wherein M is as detailed hereinafter for the halogenide of formula MYᵢ·jH₂O, such as ZnO or ZrO₂. The catalyst is preferably a Lewis acid, especially a halogenide of formula MYᵢ·jH₂O wherein M is an element selected from the group consisting of beryllium, transition metals, post-transition metals and metalloids, wherein Y is a halogen, i is the valence of element M and j is an integer ranging generally from 0 to 6, preferably 0. Transition metals are all the elements of Groups 3 to 12 of the periodic table of the elements. As herein used, post-transition metals denote the set of elements consisting of Al, Ga, In, TI, Sn, Pb, Bi and Po, while metalloids denote the set of elements consisting of B, Si, Ge, As, Sb and Te. The element M has generally an electronegativity of from 1.3 to 1.9, preferably of from 1.5 to 1.8, possibly of 1.5 or 1.6, and is more preferably Be, Ti, Sn, Al, Fe or Zn. As examples of suitable catalysts of formula MYᵢ·jH₂O, the following anhydrous halogenides (j=0) can be cited: BeCl₂, BF₃, TiCl₄, SbCl₅, SnCl₄, AlCl₃, FeCl₃ and ZnCl₂. Good results were obtained when using anhydrous aluminum trichloride (AlCl₃) as the catalyst. Good results can also be obtained when using anhydrous iron trichloride (FeCl₃) as the catalyst.

The total amount of the catalyst, based on the total combined amount of the aromatic compound A and the hexachloroxylene compound, ranges usually from 0.3 to 30 mol/mol, preferably from 1 to 10 mol/mol and more preferably from 2 to 5 mol/mol.

The whole amount of the catalyst is advantageously present in the reaction medium before the introduction of the aromatic compound A and the introduction of the hexachloroxylene compound have started.

The reaction medium can further comprise a co-catalyst. The co-catalyst can further increase the cyclization reaction rate between the aromatic compound A of formula (II) and the hexachloroxylene compound of formula (III). The co-catalyst may be a complexing agent capable of forming a complex with the catalyst. The co-catalyst may be a Lewis base. The co-catalyst is advantageously selected from the set consisting of dialkylsulfoxides, tri-, tetra-, penta- and hexa-alkylphosphoramides, mono-, di-, tri- and tetra-alkylureas, N-alkylalkanamides, N,N-dialkylalkanamides, pyrrolidone and N-alkyl-2-pyrrolidones. For all the compounds of this set of possible co-catalysts which contain one or more alkyl substituents, the at least one alkyl substituent is preferably C₁-C₈ alkyl, more preferably C₁-C₄ alkyl, still more preferably methyl or ethyl, the most preferably methyl. As examples of suitable co-catalysts, it can be cited dimethylsulfoxide, hexamethylphosphoramide, tetramethylurea, N-methylformamide, N,N-dimethylformamide, N,N-dimethylacetamide, pyrrolidone and N-methylpyrrolidone. The co-catalyst is preferably pyrrolidone or a N-alkyl-2-pryrrolidone, more preferably a N—C₁-4 alkyl-2-pyrrolidone, still more preferably N-methyl-2-pyrrolidone.

The total amount of the catalyst, based on the total combined amount of the aromatic compound A and the hexachloroxylene compound, ranges usually from 0.1 to 10 mol/mol, preferably from 0.3 to 3 mol/mol and more preferably from 0.5 to 2 mol/mol.

The whole amount of the co-catalyst is advantageously present in the reaction medium before the introduction of the aromatic compound A and the introduction of the hexachloroxylene compound have started.

Before the introduction of the aromatic compound A and the introduction of the hexachloroxylene compound have started, the reaction medium may consist of the solvent, may be composed of the solvent and the catalyst or may be composed of the solvent, the catalyst and the co-catalyst. Alternatively, the reaction medium may comprise, in addition to the solvent, in addition to the solvent and the catalyst or in addition to the solvent, the catalyst and the co-catalyst, at least one other ingredient such as a diluent, a viscosity modifier or a heat stabilizer.

Advantageously, the step S¹ takes place at a temperature of from 0 to 120° C., preferably of from 10° C. to 100° C. and more preferably of from 15° C. to 80° C. (possibly at room temperature, typically from 15° C. to 35° C.). Advantageously, the step S¹ takes place at a pressure of from 0.1 to 10 bar, preferably from 0.8 to 1.2 bar and still more preferably at the atmospheric pressure (about 101325 Pa).

The step S¹ takes advantageously place under stirring.

During the step S¹ the reaction medium may be in contact with air or under an inert atmosphere, such as a nitrogen or argon atmosphere.

When the cyclic oligo(arylene ether ketone) of formula (V) is the desired product, the invented process further comprises the step S². During the step S², the oligo(dichloromethylene arylene ether) of formula (IV) obtained during the step S¹ is advantageously hydrolyzed by a hydrolyzing agent.

The process P¹ according to the present invention may not comprise the step S². For example, the oligo(dichloromethylene arylene ether) of formula (IV) can be valorized as such as a component of various compositions, or it can be polymerized in accordance with the process P² to obtain an acyclic poly(dichloromethylene arylene ether); the acyclic poly(dichloromethylene arylene ether) can in turn be advantageously used for the manufacture of an acyclic poly(arylene ether ketone) in accordance with the method M⁴.

Preferably, the process P¹ comprises the step S².

The step S² begins generally when at least part of the hydrolyzing agent is introduced in the reaction medium.

The hydrolyzing agent is advantageously water, but other Lewis bases compounds of O in oxidation state −2 such as OH and C₁-C₄ dialkyl ethers can work.

The amount r_{H} of the hydrolyzing agent, based on the total combined amount of the aromatic compound A and the hexachloroxylene compound used during the step S¹, can vary to a large extent, depending on the choice of the hydrolyzing agent. r_{H} is usually of at least 0.80 mol/mol, preferably of at least 1 mol/mol; it may be well above 1 and even well above 10 mol/mol, e.g. about 50, especially when H₂O is used as the hydrolyzing agent.

Once the whole amount of the hydrolyzing agent has been introduced in the reaction medium, the oligo(dichloromethylene arylene ether) of formula (IV) and the hydrolysis agent are advantageously reacted for at least 30 min, preferably for at least 1 h, more preferably for at least 2 h, still more preferably for at least 3 h to ensure the highest achievable conversion into the cyclic oligo(arylene ether ketone) of formula (V).

During the step S², the catalyst used during the step S¹ is also advantageously quenched by a quenching agent. Preferably, the hydrolysis of the oligo(dichloromethylene arylene ether) of formula (IV) and the quenching of the catalyst are caused to happen concomitantly.

The nature of the quenching agent is not particularly limited provided it can quench the catalytic action of the catalyst. The quenching agent is advantageously selected from the group consisting of water and Bronsted acids such as hydrochloric acid, sulfuric acid, nitric acid and phosphoric acid.

An aqueous solution of hydrochloric acid is preferably used during the step S² as a combined hydrolyzing/quenching agent. The HCl concentration of this aqueous solution ranges usually from 0.1 to 10 mol HCl/l solution, preferably from 0.3 to 3 mol HCl/l solution and more preferably from 0.5 to 2 mol HCl/1 solution. Good results were obtained when using an about 1N HCl aqueous solution, as a combined hydrolyzing/quenching agent during the steps S².

The amount r_{Q} of hydrochloric acid or other quenching agent, based on the total combined amount of the aromatic compound A and the hexachloroxylene compound used during the step S¹, ranges usually from 0.80 to 3 mol/mol, preferably from 0.95 to 1.50 mol/mol and more preferably from 0.98 to 1.20 mol/mol. Good results were obtained when r_{Q} was about 1 mol/mol.

Advantageously, the step S² takes place at a temperature of from 0° C. to 200° ° C., preferably of from 20° ° C. to 100° C. and more preferably from 35° C. to 70° C., and at a pressure of from 0.1 bar to 10 bar, preferably from 0.8 bar to 1.2 bar and still more preferably at the atmospheric pressure (about 101325 Pa).

The step S² takes advantageously place under stirring.

During the step S², the reaction medium may be in contact with air or under an inert atmosphere, such as a nitrogen or argon atmosphere.

The cyclic oligo(arylene ether) of formula (I), viz. the cyclic oligo(dichloromethylene arylene ether) of formula (IV) or the cyclic oligo(arylene ether ketone) of formula (V), can be concentrated and/or isolated from the reaction medium using conventional concentration and isolation methods that are well-known to the skilled person. It can be among others cited liquid-liquid extraction, liquid-solid extraction, precipitation, filtration, distillation, sublimation and liquid phase chromatography.

The cyclic oligo(arylene ether) of formula (I), viz. the cyclic oligo(dichloromethylene arylene ether) of formula (IV) or the cyclic oligo(arylene ether ketone) of formula (V), can be advantageously used for the manufacture of a higher molecular weight acyclic poly(arylene ether) comprising repeat units of formula (VI), viz. a higher molecular weight acyclic poly(dichloromethylene arylene ether) comprising repeat units of formula (VII) or a higher molecular weight acyclic poly(arylene ether ketone) comprising repeat units of formula (VIII).

The higher molecular weight acyclic poly(arylene ether) comprising repeat units of formula (VI) is advantageously linear; otherwise said, it is advantageously acyclic and free of ramifications. The repeat units comprised in the higher molecular weight acyclic poly(arylene ether) can be kinked or straight.

The higher molecular weight acyclic poly(arylene ether) comprising repeat units of formula (VI) can be any of the above described acyclic poly(arylene ether)s, including the one comprising m repeat units of formula (VI), the one comprising m repeat units of formula (VII), the one comprising m repeat units of formula (VIII), the one comprising m repeat units of formula (XII), the one comprising m repeat units of formula (XIII), the one comprising m repeat units of formula (XIV), the one comprising m′ repeat units of formula (XVIII), the one comprising m′ repeat units of formula (XIX) or the one comprising m′ repeat units of formula (XX), wherein m, wherever previously used, is greater than 5 times n and is possibly greater than 50 times n, with n as previously defined for the cyclic oligo(arylene ether) of formula (I), and wherein m′, wherever previously used, is greater than 10. m and m′ may be of at least 20, 50, 100, 150 or 200. m and m′ are generally of at most 500 or 1000. It can be a copolymer or a homopolymer.

It can comprise the repeat units of formula (VI), (VII), (VIII), (XII), (XIII), (XIV), (XVIII), (XIX) or (XX), as the case may be, in a weight amount which exceeds advantageously 50%, preferably 90%, more preferably 99% of the total weight of its repeat units. Still more preferably, it comprises, as sole repeat units, repeat units selected from the group consisting of the repeat units of formulae (VI), (VII), (VIII), (XII), (XIII), (XIV), (XVIII), (XIX) and (XX). The most preferably, it is a PEKK homo- or co-polymer comprising as sole repeat units, repeat units of formula (XXVI)

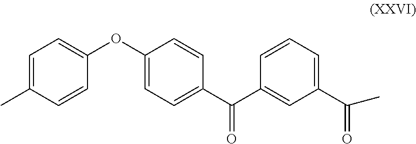

and/or repeat units of formulae (XXVII)

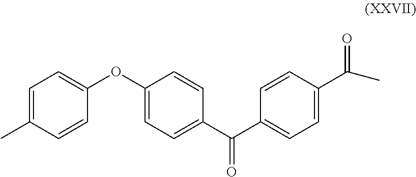

Of most industrial importance are the PEKK copolymers comprising, as sole repeat units, from 15 to 50 wt. % of repeat units of formula (XXVI) and from 85 to 50 wt. % of repeat units of formula (XXVII).

The higher molecular weight acyclic poly(arylene ether) comprising repeat units of formula (VI) can be notably prepared by the process P², by the method M², by the method M³ or by the method M⁴.

The ring-opening polymerization of the cyclic oligo(arylene ether) of formula (I) can take place “in solution”, that is to say in a polymerization medium comprising a solvent which dissolves the cyclic oligo(arylene ether) of formula (I) and the higher molecular weight acyclic poly(arylene ether) comprising repeat units of formula (VI). Alternatively, the ring-opening polymerization of the cyclic oligo(arylene ether) of formula (I) can take place “in the melt”, that is to say in a polymerization medium wherein the cyclic oligo(arylene ether) of formula (I) and the higher molecular weight acyclic poly(arylene ether) comprising repeat units of formula (VI) are at molten state. When the cyclic oligo(arylene ether) of formula (I) is a cyclic oligo(arylene ether ketone), the ring-opening polymerization takes preferably place in the melt; this is especially true when the cyclic oligo(arylene ether ketone) is impregnated on a continuous fiber.

The ring-opening polymerization of the cyclic oligo(arylene ether) of formula (I) takes advantageously place in the presence of an initiator, preferably cesium fluoride, a phenolate (e.g. potassium biphenylbisphenolate) or a sodium, potassium or cesium salt of 4-hydroxybenzophenone.

The initiator:cyclic oligo(arylene ether) molar ratio is generally 0.001-0.05:1.

The ring-opening polymerization takes usually place at a temperature from 100° C. to 450° C. It is preferably of at least 200° C., more preferably of at least 300° C. Besides, it is preferably of at most 350° C.

The ring-opening polymerization can take place in an inert atmosphere, under vacuum or under air.

The ring-opening polymerization can be carried out in an extruder.

When a composite material comprising a poly(arylene ether ketone) and a continuous fiber is formed, the ring-opening polymerization is preferably carried out in an extruder or during the continuous fiber impregnation. The continuous fiber impregnation can be notably melt impregnation, solution impregnation or slurry impregnation.

The initiator and cyclic oligo(arylene ether) are advantageously heated until the polymerization temperature is reached.

Then, the ring-opening polymerization is allowed to proceed generally for 0.1-600 min, preferably for 2-180 min and more preferably for 3-60 min, so as to obtain the higher molecular weight acyclic poly(arylene ether). If the ring-opening polymerization is carried out in an extruder, the ring-opening polymerization is typically allowed to proceed for a duration which is lower than or equal to the residence time of the reaction mixture in the extruder, which may be very short, possibly below 10 min. Otherwise, longer durations may be applied, for example of at least 10, at least 20 or at least 30 min.

A transfer agent, such as monofluorobenzophenone, monochlorobenzophenone or monochlorodiphenylsulfone, may be added in the polymerization medium, possibly at start or during the course of the ring-opening polymerization.

When a composite material is manufactured in accordance with the present invention, the continuous fiber is advantageously carbon fiber or glass fiber, preferably carbon fiber.

Composite materials incorporating a PEKK homo- or co-polymer [especially, a PEKK copolymer comprising, as sole repeat units, from 15 to 50 wt. % of meta repeat units of formula (XXVI) and from 85 to 50 wt. % of para repeat units of formula (XXVII)], and a continuous fiber (such as glass fiber or carbon fiber), are worth being cited as high-performance composite materials of particular industrial importance that can be made in accordance with the process P² and the uses U¹, U² and U³ of the present invention.

In the use U², the para-aromatic cyclic oligo(arylene ether) of formula (IX) can be of formula (X) or (XI).

In the use U³, the meta-aromatic cyclic di(arylene ether) of formula (XV) can be of formula (XVI) or (XVII).

The present invention has numerous advantages.

It enables the manufacture of cyclic oligo(arylene ether ketone) products with a high yield, higher than the one obtained by Changchun, in particular when meta-aromatic cyclic oligo(arylene ether ketone)s are desired. In contrast with Changchun's process, the cyclic oligo(arylene ether ketone)s are manufactured without generating a substantial amount of insoluble by-products: at the end of the cyclization reaction, the reaction medium of the present invention is substantially free, essentially free or even completely free of insoluble by-products.

It makes it possible to manufacture new para-aromatic cyclic oligo(arylene ether ketone)s, especially new para-aromatic cyclic oligo(arylene ether ketone) dimers, with a high yield and selectivity. It makes it also possible to manufacture new meta-aromatic cyclic oligo (dichloromethylene arylene ether) dimers with a high yield and selectivity.

The cyclic oligo(arylene ether ketone)s of the present invention can be polymerized by ring-opening polymerization, with a wide processing window for the preparation of composite materials. This makes the ring-forming reaction route have an important application prospect in the field of preparing high-performance composite materials. This is especially true for the dimers which have a somewhat lower melt viscosity than their higher-size homologues (trimers, tetramers, etc.) Composite materials incorporating a PEKK homo- or co-polymer (especially, a PEKK copolymer comprising, as sole repeat units, from 15 to 50 wt. % of meta repeat units and from 85 to 50 wt. % of para repeat units), and a continuous fiber such as glass fiber or carbon fiber, are worth being cited as high-performance composite materials of particular industrial importance that can be made from the cyclic oligo(arylene ether ketone)s of the present invention.

It provides also new cyclic oligo(dichloromethylene arylene ether)s, in particular new meta-aromatic and new para-aromatic cyclic oligo(dichloromethylene arylene ether)s, including dimers, and a process for the manufacture with a high yield and selectivity. These ones can be used for the manufacture of high molecular weight acyclic, generally linear, poly(dichloromethylene arylene ether)s, which can be hydrolyzed, if desired, in the corresponding poly(arylene ether ketone)s.

For the avoidance of doubt, wherever used throughout the present patent title,

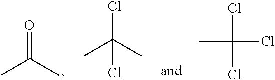

moieties represent respectively carbonyl

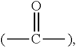

dichloromethylene (—CCl₂—) and trichloromethyl (—CCl₃) moieties.

For the avoidance of doubt, wherever used throughout the present patent title,

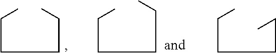

representations and the like, which are used in formulae

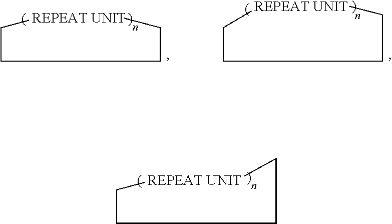

and the like of cyclic oligo(arylene ether)s, represent invariably a direct single bond — which “closes the loop” of the cyclic oligo(arylene ether)s by connecting two of their repeat units. For example, in the cyclic oligo(arylene ether) of formula (I),

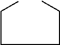

represents a direct single bond — which “closes the loop” by connecting a carbon atom of a benzene ring to a L′ moiety; it does not represent a —CH₂—CH₂—CH₂—CH₂-moiety.

Wherever used throughout the present patent title, the indefinite article “a” is intended to encompass both the singular and the plural forms of the noun to which it relates, and has thus the same meaning as “at least one”. Likewise, the definite article “the” is intended to encompass both the singular and the plural forms of the noun to which it relates, and has thus the same meaning as “the at least one”.

Should the disclosure of any patents, patent applications, and publications which are incorporated herein by reference conflict with the description of the present application to the extent that it may render a term unclear, the present description shall take precedence.

The present invention will now be illustrated by the following examples, which are not intended to be limiting.

### EXAMPLES

### Example 1 (for Comparison Purposes)

In a 250 ml double-jacket reactor under N₂ were charged successively 1,2-dichloroethane (100 ml), AlCl₃ (2 g, 15 mmol, 6 eq.), 1,2-dichloroethane again (25 ml) and N-methyl-2-pyrrolidone (0.248 g, 0.241 ml, 2.5 mmol, 1 eq.), so as to form a yellow mixture 1. The yellow mixture 1 was stirred at 300 rpm at 25° C. for 10 min. Then, a mixture 2 of isophthaloyl chloride (0.508 g, 2.5 mmol, 1 eq.) and diphenyl ether (0.426 g, 0.397 ml, 2.5 mmol, 1 eq.) in 1,2-dichloroethane (25 ml) was added slowly over a period of time of 8 hours to the yellow mixture 1, so as to form a reactive mixture 3. The addition rate of isophthaloyl chloride and diphenyl ether was kept approximately constant during this 8-hour period of time, and equal to about 1.058 mg/min of isophthaloyl chloride and about 0.8875 mg/min of diphenyl ether. During the 8-hour period of time, the mixture 3 turned progressively from yellow to deep red, and an insoluble solid by-product 4 appeared during this period. Then, 1N HCl (50 ml) was added to the deep red mixture 3, which comprised cyclic oligo(phenylene ether ketone)s and unreacted AlCl₃, so as to form a new reactive mixture 5. The reactive mixture 5 was stirred at 50° C. for 4 hours, so as to allow for the quenching of unreacted AlCl₃. After 4 hours, a triphasic mixture 6 consisting of an organic phase 6a comprising cyclic oligo(phenylene ether ketone)s, an aqueous phase 6b comprising quenched AlCl₃, and the insoluble solid by-product 4 was obtained. The triphasic mixture 6 was cooled to 25° C. and filtered to remove the insoluble solid by-product 4. The filtrate recovered after filtration consisting of the organic phase 6a and the aqueous phase 6b was left to settle during 5 min in a separating funnel, with the organic phase 6a at its bottom and the aqueous phase 6b at its top. The phases 6a and 6b were separately drawn off from the separating funnel. About 20 ml of dichloromethane were added to the aqueous phase 6b under stirring to form a biphasic mixture 7 comprising an organic phase 7a and an aqueous phase 7b. The biphasic mixture 7 was left to settle during 5 min in a separating funnel, with the organic phase 7a at its bottom and the aqueous phase 7b at its top. The phases 7a and 7b were separately drawn off from the separating funnel. Again, about 20 ml of dichloromethane were added to the aqueous phase 7b under stirring to form a new biphasic mixture 8 comprising an organic phase 8a and an aqueous phase 8b. The biphasic mixture 8 was in turn left to settle during 5 min in a separating funnel, with the organic phase 8a at its bottom and the aqueous phase 8b at its top. The phases 8a and 8b were separately drawn off from the separating funnel. The organic phases 6a, 7a and 8a were mixed together so as to form an organic phase 9. The organic phase 9, which contained cyclic oligo(phenylene ether ketone)s and HCl, was washed with about 20 ml of water under stirring to form a biphasic mixture 10, with an organic phase 10a and an aqueous phase 10b. The biphasic mixture 10 was left to settle during 5 min in a separating funnel, with the organic phase 10a at its bottom and the aqueous phase 10b at its top. The pH of the aqueous phase 10b was measured; it was well below 4. Again, about 20 ml of water were added to the organic phase 10a under stirring to form a biphasic mixture 11 with an organic phase 11a and an aqueous phase 11b. The biphasic mixture 11 was in turn left to settle during 5 min in a separating funnel, with the organic phase 11a at its bottom and the aqueous phase 11b at its top. The pH of the aqueous phase 11b was measured; it was substantially above 4. Should the pH had still been below 4, a further wash with H₂O could have been desirable to remove further HCl residues from the organic phase 11a. Then, 5 g of anhydrous MgSO₄ were added under stirring to the organic phase 11a which comprised a residual amount of water. The so-obtained MgSO₄-organic phase admixture 12 was then filtered using a sintered filter, which retained hydrated MgSO₄ on the filter. An organic filtrate 13 essentially free of water was recovered; it comprised cyclic oligo(phenylene ether ketone)s, 1,2-dichloroethane and dichloromethane as main components. The 1,2-dichloroethane and dichloromethane were then evaporated from the organic filtrate 13 using a Buchi rotary evaporator at a temperature of 40° ° C. and under reduced pressure (300 mbar), so as to afford 415 mg of a green solid 14 comprising cyclic oligo(phenylene ether ketone)s, corresponding to a yield of 55%, as defined by the ratio of the weight of the green solid 14 to the theoretical weight of oligo(phenylene ether ketone) repeat units obtainable by the full conversion of isophthaloyl chloride and diphenyl ether starting reagents into such oligo(phenylene ether ketone) repeat units.

### Characterization of the Green Solid 14

### HPLC

The HPLC chromatogram showed the presence of cyclic oligo(phenylene ether ketone)s, more precisely of:
cyclic PEKK trimer having a retention time of 3.7 min,
cyclic PEKK tetramer having a retention time of 5.3 min,
cyclic PEKK pentamer having a retention time of 6.4 min,
cyclic PEKK hexamer having a retention time of 7.3 min, and
cyclic PEKK heptamer having a retention time of 7.9 min.

The distribution of the cyclic PEKK oligomers, estimated from the respective surface areas they develop on the HPLC chromatogram, is as follows:
47% of cyclic PEKK trimer,
31% of cyclic PEKK tetramer,
15% of cyclic PEKK pentamer,
5% of cyclic PEKK hexamer, and
2% of cyclic PEKK heptamer.

To the contrary, no cyclic PEKK dimer, which would have had a retention time of about 1.9-2.2 min, was identified.

### NMR

Green solid 14 (1H, CDCl₃): 7.9-8.2 ppm (m, 2H), 7.7-7.9 ppm (m, 4H), 7.5-7.65 ppm (m, 2H), 6.9-7.3 ppm (m, 4H).

### Example 2 (According to the Invention)

In a 250 ml double-jacket reactor under N₂ were charged successively 1,2-dichloroethane (100 ml), AlCl₃ (2 g, 15 mmol, 6 eq.), 1,2-dichloroethane again (25 ml) and N-methyl-2-pyrrolidone (0.248 g, 0.241 ml, 2.5 mmol, 1 eq.), so as to form a yellow mixture 21. The yellow mixture 21 was stirred at 300 rpm at 25° C. for 10 min. Then, a mixture 22 of hexachlorometaxylol (0.782 g, 2.5 mmol, 1 eq.) and diphenyl ether (0.426 g, 0.397 ml, 2.5 mmol, 1 eq.) in 1,2-dichloroethane (25 ml) was added slowly over a period of time of 8 hours to the yellow mixture 21, so as to form a reactive mixture 23. The addition rate of hexachlorometaxylol and diphenyl ether was kept approximately constant during this 8-hour period of time, and equal to about 1.629 mg/min of hexachlorometaxylol and about 0.8875 mg/min of diphenyl ether. During the 8-hour period of time, the mixture 23 turned progressively from yellow to deep red. Then, 1N HCl (50 ml) was added to the deep red mixture 23, which comprised cyclic oligo(dichloromethylene phenylene ether)s and unreacted AlCl₃, so as to form a new reactive mixture 24. The reactive mixture 24 was stirred at 50° C. for 4 hours, so as to allow for the hydrolysis of cyclic oligo(dichloromethylene phenylene ether)s into the corresponding cyclic oligo(phenylene ether ketone)s and for the quenching of unreacted AlCl₃. After 4 hours, a biphasic mixture 25 consisting of an organic phase 25a comprising cyclic oligo(phenylene ether ketone)s and an aqueous phase 25b comprising quenched AlCl₃ was obtained. The biphasic mixture 25 was cooled to 25° C. and left to settle during 5 min in a separating funnel, with the organic phase 25a at its bottom and the aqueous phase 25b at its top. The phases 25a and 25b were separately drawn off from the separating funnel. About 20 ml of dichloromethane were added to the aqueous phase 25b under stirring to form a new biphasic mixture 26 comprising an organic phase 26a and an aqueous phase 26b. The biphasic mixture 26 was left to settle during 5 min in a separating funnel, with the organic phase 26a at its bottom and the aqueous phase 26b at its top. The phases 26a and 26b were separately drawn off from the separating funnel. Again, about 20 ml of dichloromethane were added to the aqueous phase 26b under stirring to form a new biphasic mixture 27 comprising an organic phase 27a and an aqueous phase 27b. The biphasic mixture 27 was in turn left to settle during 5 min in a separating funnel, with the organic phase 27a at its bottom and the aqueous phase 27b at its top. The phases 27a and 27b were separately drawn off from the separating funnel. The organic phases 25a, 26a and 27a were mixed together so as to form an organic phase 28. No trace of solid impurities was observed. The organic phase 28, which contained cyclic oligo(phenylene ether ketone)s and HCl, was washed with about 20 ml of water under stirring to form a biphasic mixture 29, with an organic phase 29a and an aqueous phase 29b. The biphasic mixture 29 was left to settle during 5 min in a separating funnel, with the organic phase 29a at its bottom and the aqueous phase 29b at its top. The pH of the aqueous phase 29b was measured; it was well below 4. Again, about 20 ml of water were added to the organic phase 29a under stirring to form a biphasic mixture 30, with an organic phase 30a and an aqueous phase 30b. The biphasic mixture 30 was in turn left to settle during 5 min in a separating funnel, with the organic phase 30a at its bottom and the aqueous phase 30b at its top. The pH of the aqueous phase 30b was measured; it was substantially above 4. Should the pH had still been below 4, a further wash with H₂O could have been desirable to remove further HCl residues from the organic phase 30a. Then, 5 g of anhydrous MgSO₄ were added under stirring to the organic phase 30a which comprised a residual amount of water. The so-obtained MgSO₄-organic phase admixture 31 was then filtered using a sintered filter, which retained hydrated MgSO₄ on the filter. An organic filtrate 32 essentially free of water was recovered; it comprised cyclic oligo(phenylene ether ketone)s, 1,2-dichloroethane and dichloromethane as main components. The 1,2-dichloroethane and dichloromethane were then evaporated from the organic filtrate 32 using a Buchi rotary evaporator at a temperature of 40° C. and under reduced pressure (300 mbar), so as to afford 694 mg of a brown foam 33 comprising cyclic oligo(phenylene ether ketone)s, corresponding to a yield of 93%, as defined by the ratio of the weight of the brown foam 33 to the theoretical weight of oligo(phenylene ether ketone) repeat units obtainable by the full conversion of hexachlorometaxylol and diphenyl ether starting reagents into such oligo(phenylene ether ketone) repeat units.

### Characterization of the Brown Foam 33

### HPLC

The HPLC chromatogram showed the presence of cyclic oligo(phenylene ether ketone)s, more precisely of:
cyclic PEKK dimer having a retention time of 1.9 min,
cyclic PEKK trimer having a retention time of 3.7 min,
cyclic PEKK tetramer having a retention time of 5.3 min, and
cyclic PEKK pentamer having a retention time of 6.4 min.

The distribution of the cyclic PEKK oligomers, estimated from the respective surface areas they develop on the HPLC chromatogram, is as follows:
50% of cyclic PEKK dimer,
29% of cyclic PEKK trimer,
14% of cyclic PEKK tetramer, and
7% of cyclic PEKK pentamer.

### NMR

Brown foam 13 (1H, CDCl₃): 6.9-8.4 ppm (m, 12H).

### Example 3 (According to the Invention)

In a 250 ml double-jacket reactor under N₂ were charged successively 1,2-dichloroethane (100 ml), AlCl₃ (2 g, 15 mmol, 6 eq.), 1,2-dichloroethane again (25 ml) and N-methyl-2-pyrrolidone (0.248 g, 0.241 ml, 2.5 mmol, 1 eq.), so as to form a yellow mixture 41. The yellow mixture 41 was stirred at 300 rpm at 25° ° C. for 10 minutes. Then, a mixture 42 of hexachloroparaxylol (0.782 g, 2.5 mmol, 1 eq.) and diphenyl ether (0.426 g, 0.397 ml, 2.5 mmol, 1 eq.) in 1,2-dichloroethane (25 ml) was added slowly over a period of time of 8 hours to the yellow mixture 41, so as to form a reactive mixture 43. The addition rate of hexachloroparaxylol and diphenyl ether was kept approximately constant during this 8-hour period of time, and equal to about 1.629 mg/min of hexachloroparaxylol and about 0.8875 mg/min of diphenyl ether. During the 8-hour period of time, the mixture 43 turned progressively from yellow to deep red. Then, 1N HCl (50 ml) was added to the deep red mixture 43, which comprised cyclic oligo(dichloromethylene phenylene ether)s and unreacted AlCl₃, so as to form a new reactive mixture 44. The reactive mixture 44 was stirred at 50° C. for 4 hours, so as to allow for the hydrolysis of cyclic oligo(dichloromethylene phenylene ether)s into the corresponding cyclic oligo(phenylene ether ketone)s and for the quenching of unreacted AlCl₃. After 4 hours, a biphasic mixture 45 consisting of an organic phase 45a comprising cyclic oligo(phenylene ether ketone)s and an aqueous phase 45b comprising quenched AlCl₃ was obtained. The biphasic mixture 45 was cooled to 25° C. and left to settle during 5 min in a separating funnel, with the organic phase 45a at its bottom and the aqueous phase 45b at its top. The phases 45a and 45b were separately drawn off from the separating funnel. About 20 ml of dichloromethane were added to the aqueous phase 45b under stirring to form a new biphasic mixture 46 comprising an organic phase 46a and an aqueous phase 46b. The biphasic mixture 46 was left to settle during 5 min in a separating funnel, with the organic phase 46a at its bottom and the aqueous phase 46b at its top. The phases 46a and 46b were separately drawn off from the separating funnel. Again, about 20 ml of dichloromethane were added to the aqueous phase 46b under stirring to form a new biphasic mixture 47 comprising an organic phase 47a and an aqueous phase 47b. The biphasic mixture 47 was in turn left to settle during 5 min in a separating funnel, with the organic phase 47a at its bottom and the aqueous phase 47b at its top. The phases 47a and 47b were separately drawn off from the separating funnel. The organic phases 45a, 46a and 47a were mixed together so as to form an organic phase 48. The organic phase 48, in which a few traces of insoluble impurities were observed, was filtrated on a sintered filter. An organic filtrate 49 was recovered; it comprised cyclic oligo(phenylene ether ketone)s and HCl. About 20 ml of water were added to the organic filtrate 49 under stirring to form a biphasic mixture 50, with an organic phase 50a and an aqueous phase 50b. The biphasic mixture 50 was left to settle during 5 min in a separating funnel, with the organic phase 50a at its bottom and the aqueous phase 50b at its top. The pH of the aqueous phase 50b was measured; it was well below 4. Again, about 20 ml of water were added to the organic phase 50a under stirring to form a biphasic mixture 51, with an organic phase 51a and an aqueous phase 51b. The biphasic mixture 51 was in turn left to settle during 5 min in a separating funnel, with the organic phase 51a at its bottom and the aqueous phase 51b at its top. The pH of the aqueous phase 51b was measured; it was substantially above 4. Should the pH had still been below 4, a further wash with H₂O could have been desirable to remove further HCl residues from the organic phase 51a. Then, 5 g of anhydrous MgSO₄ were added under stirring to the organic phase 51a which comprised a residual amount of water. The so-obtained MgSO₄-organic phase admixture 52 was then filtered using a sintered filter, which retained hydrated MgSO₄ on the filter. An organic filtrate 53 essentially free of water was recovered; it comprised cyclic oligo(phenylene ether ketone)s, 1,2-dichloroethane and dichloromethane as main components. The 1,2-dichloroethane and dichloromethane were then evaporated from the organic filtrate 53 using a Buchi rotary evaporator at a temperature of 40° C. and under reduced pressure (300 mbar), so as to afford 645 mg of a brown foam 54 comprising cyclic oligo(phenylene ether ketone)s, corresponding to a yield of 86%, as defined by the ratio of the weight of the brown foam 54 to the theoretical weight of oligo(phenylene ether ketone) repeat units obtainable by the full conversion of hexachloroparaxylol and diphenyl ether starting reagents into such oligo(phenylene ether ketone) repeat units.

### Characterization of the Brown Foam 54

### HPLC

The HPLC chromatogram showed the presence of cyclic oligo(phenylene ether ketone)s, more precisely of:
cyclic PEKK dimer having a retention time of 2.2 min,
cyclic PEKK trimer having a retention time of 3.9 min,
cyclic PEKK tetramer having a retention time of 4.9 min, and
cyclic PEKK pentamer having a retention time of 6.12 min.

The distribution of the cyclic PEKK oligomers, estimated from the respective surface areas they develop on the HPLC chromatogram, is as follows:
34% of cyclic PEKK dimer,
34% of cyclic PEKK trimer,
19% of cyclic PEKK tetramer, and
13% of cyclic PEKK pentamer.

### NMR

Brown foam 54 (1H, CDCl₃): 6.8-8.0 ppm (m, 12H).

1. A process P¹ for the manufacture of a cyclic oligo(arylene ether) of formula (I) 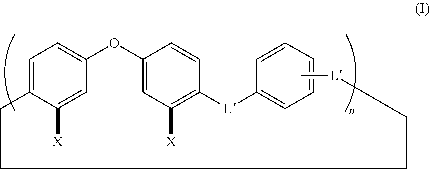
wherein
X, which is the same or different at each occurrence, is hydrogen or methyl,
L, which is the same or different at each occurrence, is a divalent moiety selected from the group consisting of
—O—, —CX₂— with X as previously defined, 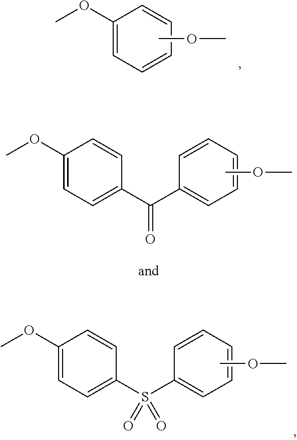 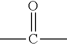
L′, which is the same at each occurrence, is a —CCl₂— or divalent moiety, and
n is an integer ranging from 2 to 20,
said process comprising a step S¹ of causing an aromatic compound A of formula (II) 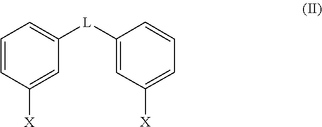
wherein X and L are as defined for the cyclic oligo(arylene ether) of formula (I), to react with a hexachloroxylene compound of formula (III) 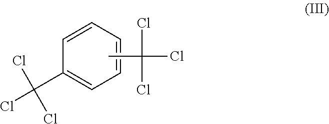
in a reaction medium comprising a solvent S capable of dissolving the cyclic oligo(arylene ether), the aromatic compound A and the hexachloroxylene compound,
wherein the reaction medium is a pseudo-high dilution environment.

2. The process according to claim 1, wherein the aromatic compound A, the hexachloroxylene compound, or both are introduced progressively in the reaction medium.

3. The process according to claim 1, wherein, when the period of time I¹ enveloping introduction of a whole amount of the aromatic compound A and a whole amount of the hexachloroxylene compound in the reaction medium, is divided in k=8 parts of equal duration D¹/k, D¹ being the duration of I¹, at least half of k average introduction rates of the aromatic compound A r_{avg.A}, r²_{avg.A}, . . . r^{k}_{avg.A} do not exceed a certain value T_{A}^{max}=10 mmol/(l·h), at least half of the k average introduction rates of the hexachloroxylene compound r_{avg.B}, r²_{avg.B}, . . . r^{k}_{avg.B} do not exceed a certain value r_{B}^{max}=10 mmol/(l·h), or both.

4. The process according to claim 1, wherein a cyclic oligo(dichloromethylene arylene ether) of formula (IV) 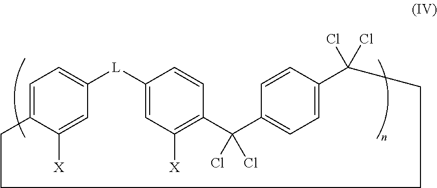
wherein X, L and n are as defined for the cyclic oligo(arylene ether) of formula (I),
is obtained as a reaction product of the reaction which takes place during the step S¹, and wherein the process further comprises a step S² wherein the obtained cyclic oligo(dichloromethylene arylene ether) of formula (IV) is hydrolyzed, so as to obtain a cyclic oligo(arylene ether ketone) of formula (V) 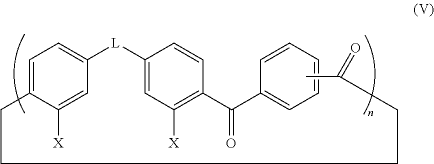
wherein X, L and n are as defined for the cyclic oligo(arylene ether) of formula (I).

5. A process P² for the manufacture of an acyclic poly(arylene ether) comprising m repeat units of formula (VI) 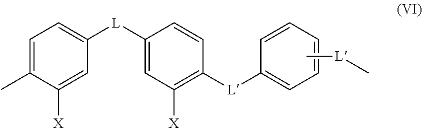
wherein m is an integer which is greater than 5 times n
said process comprising the steps of:
manufacturing the cyclic oligo(arylene ether) of formula (I) by the process P¹ according to claim 1, and
causing the cyclic oligo(arylene ether) of formula (I) to undergo a ring-opening polymerization, so as to obtain the poly(arylene ether) comprising m repeat units of formula (VI).

6. The process according to claim 5, wherein the acyclic poly(arylene ether) comprising m repeat units of formula (VI) is an acyclic poly(arylene ether ketone) comprising m repeat units of formula (VIII) 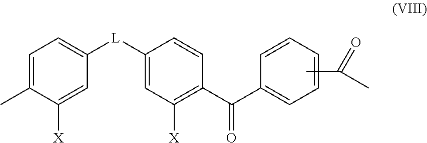
wherein m is an integer which is greater than 5 times n,
wherein the acyclic poly(arylene ether ketone) comprising m repeat units of formula (VIII) is comprised in a composite material,
said process comprising the steps of:
manufacturing the cyclic oligo(arylene ether ketone) of formula (V) by the process P¹ according to claim 4,
impregnating a continuous fiber with the cyclic oligo(arylene ether ketone) of formula (V), so as to form a pre-composite material, and
causing the cyclic oligo(arylene ether ketone) of formula (V) which is comprised in the pre-composite material to undergo a ring-opening polymerization, so as to form the composite material comprising the acyclic poly(arylene ether ketone) comprising m repeat units of formula (VIII).

7. The process according to claim 1, wherein X is hydrogen,
L is —O— and L′ is —CCl₂—.

8. The process according to claim 1, wherein X is hydrogen,
L is —O— and L′ is

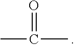

9. The process according to claim 1 wherein n ranges from 2 to 6.

10. A cyclic oligo(dichloromethylene arylene ether) of formula (IV) 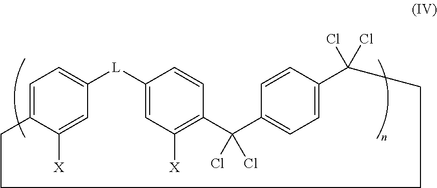
wherein X, L and n are as defined in claim 1.

11. The cyclic oligo(dichloromethylene arylene ether) according to claim 10 wherein X is hydrogen, L is —O— and n ranges from 2 to 6.

12. A method M¹ for the manufacture of a cyclic oligo(arylene ether ketone) of formula (V) 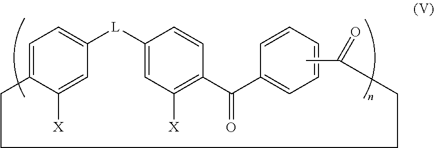
said method M¹ comprising hydrolysis of the cyclic oligo(dichloromethylene arylene ether) of formula (IV) according to claim 10 into the cyclic oligo(arylene ether ketone) of formula (V).

13. A para-aromatic cyclic oligo(arylene ether) of formula (IX) 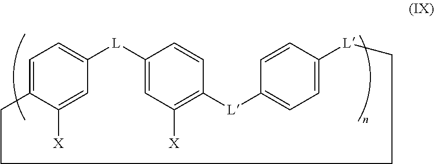
wherein X, L, L′ and n are as defined in claim 1.

14. A method M² for the manufacture of an acyclic poly(arylene ether) comprising m repeat units of formula (XII) 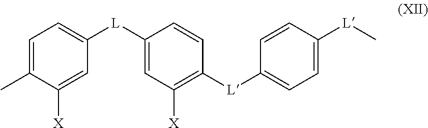
wherein m is an integer which is greater than 5 times n,
said method comprising the ring-opening polymerization of the para-aromatic cyclic oligo(arylene ether) of formula (IX) according to claim 13.

15. A meta-aromatic cyclic di(arylene ether) of formula (XV) 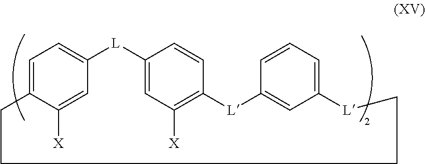
wherein X, L and L′ are as defined in claim 1.

16. A method M³ for the manufacture of an acyclic poly(arylene ether) comprising m′ repeat units of formula (XVIII) 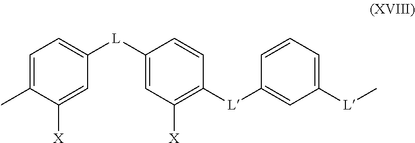
wherein m′ is an integer which is greater than 10,
said method comprising the ring-opening polymerization of the meta-aromatic cyclic di(arylene ether) of formula (XV) according to claim 15.

17. The para-aromatic cyclic oligo(arylene ether) of formula (IX) of claim 13,
wherein X is hydrogen, L is O—, L′ is 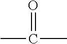
and n ranges from 2 to 6.

18. The meta-aromatic cyclic di(arylene ether) of formula (XV) of claim 15,
wherein X is hydrogen, L is O— and L′ is [chemical structure]
